(19) United States
(12) Patent Application Publication
(10) Pub. No.: US 2003/0170661 A1
(43) Pub. Date: Sep. 11, 2003

(54) Method for identifying a nucleic acid sequence

(76) Inventors: Yi Liu, San Diego, CA (US); Pascal Bouffard, Danbury, CT (US); Oswald Crasta, Clinton, CT (US)

Correspondence Address: MINTZ, LEVIN, COHN, FERRIS,, GLOVSKY and POPEO, P.C., One Financial Center, Boston, MA, 02111, US

(21) Appl. No.: 10/201,408

(22) Filed: Jul. 23, 2002

Related U.S. Application Data
(60) Provisional application No. 60/307,239, filed on Jul. 23, 2001.

(51) Int. Cl. C12Q 1/68; C12P 19/34

(52) U.S. Cl. 435/6; 435/91.2

(57) ABSTRACT

The present invention provides methods by which biologically derived DNA sequences in a mixed sample or in an arrayed single sequence clone can be determined and classified without sequencing. The methods make use of information on the presence of carefully chosen target subsequences, typically of length from 4 to 8 base pairs, the length between target subsequences, and the measurement of the presence or absence of at least one additional target subsequence in a sample DNA sequence together with DNA sequence databases containing lists of sequences likely to be present in the sample to determine a sample sequence.

### RELATED APPLICATIONS

This application claims priority to U.S. Provisional Patent Application Serial No. 60/307,239, filed Jul. 23, 2001, which is hereby incorporated in its entirety.

### FIELD OF THE INVENTION

The field of the invention is DNA sequence classification, identification or determination. More particularly it is the classification, comparison of expression, or identification of preferably all DNA sequences or genes in a sample without performing any associated sequencing.

### BACKGROUND OF THE INVENTION

As molecular biological and genetics research have advanced, it has become increasingly clear that the temporal and spatial expression of genes plays a vital role in processes occurring in both health and in disease. Moreover, the field of biology has progressed from an understanding of how single genetic defects cause the traditionally recognized hereditary disorders (e.g., the thalassemias), to a realization of the importance of the interaction of multiple genetic defects in concert with various environmental factors in the etiology of the majority of the more complex disorders, such as neoplasia.

For example, in the case of neoplasia, recent experimental evidence has demonstrated the key causative roles of multiple defects in several pivotal genes causing their altered expression. Other complex diseases have been shown to have a similar etiology. Therefore, the more complete and reliable a correlation which can be established between gene expression and disease states, the better diseases will be able to be recognized, diagnosed and treated. This important correlation may be established by the quantitative determination and classification of DNA expression in tissue samples.

Genomic DNA (“gDNA”) sequences are those naturally occurring DNA sequences constituting the genome of a cell. The overall state of gene expression within genomic DNA (“gDNA”) at any given time is represented by the composition of cellular messenger RNA (“mRNA”), which is synthesized by the regulated transcription of gDNA. Complementary DNA (“cDNA”) sequences may be synthesized by the process of reverse transcription of mRNA by use of viral reverse transcriptase. cDNA derived from cellular mRNA also represents, albeit approximately, gDNA expression within a cell at a given time. Accordingly, a methodology that would allow the rapid, economical and highly quantitative detection of all the DNA sequences within particular cDNA or gDNA samples is extremely desirable.

Heretofore, gene-specific DNA analysis methodologies have not been directed to the determination or classification of substantially all genes within a DNA sample representing the total transcribed cellular mRNA population and have universally required some degree of nucleic acid sequencing to be performed. As a result, existing cDNA and gDNA, analysis techniques have been directed to the determination and analysis of only one or two known or unknown genetic sequences at a single time. These techniques have typically utilized probes that are synthesized to specifically recognize (by the process of hybridization) only one particular DNA sequence or gene. See e.g., Watson, J. (1992) Recombinant DNA, chap 7, (W. H. Freeman, New York.). Furthermore, the adaptation of these methods to the recognition of all sequences within a sample would be, at best, highly cumbersome and uneconomical.

One existing method for detecting, isolating and sequencing unknown genes utilizes an arrayed cDNA library. From a particular tissue or specimen, mRNA is isolated and cloned into an appropriate vector, which is introduced into bacteria (e.g., E. coli) through the process of transformation. The transformed bacteria are then plated in a manner such that the progeny of individual vectors bearing the clone of a single cDNA sequence can be separately identified. A filter “replica” of such a plate is then probed (often with a labeled DNA oligomer selected to hybridize with the cDNA representing the gene of interest) and those bacteria colonies bearing the cDNA of interest are identified and isolated. The cDNA is then extracted and the insert contained therein is subjected to sequencing via protocols that includes, but are not limited to the dideoxynucleotide chain termination method. See Sanger, F. et al. (1977) DNA Sequencing with Chain Terminating Inhibitors, Proc. Natl Acad. Sci. USA 74(12):5463-5467.

The oligonucleotide probes utilized in colony selection protocols for unknown gene(s) are synthesized to hybridize, preferably, only with the cDNA for the gene of interest. One method of achieving this specificity is to start with the protein product of the gene of interest. If a partial sequence (ie., from a peptide fragment containing 5 to 10 amino acid residues) from an active region of the protein of interest can be determined, a corresponding 15 to 30 nucleotide (nt) degenerate oligonucleotide can be synthesized which would code for this peptide fragment. Thus, a collection of degenerate oligonucleotides will typically be sufficient to uniquely identify the corresponding gene. Similarly, any information leading to 15-30 nt subsequences can be used to create a single gene probe.

Another existing method, which searches for a known gene in cDNA or gDNA prepared from a tissue sample, also uses single-gene or single-sequence oligonucleotide probes that are complementary to unique subsequences of the already known gene sequences. For example, the expression of a particular oncogene in sample can be determined by probing tissue-derived cDNA with a probe that is derived from a subsequence of the oncogene's expressed sequence tag. The presence of a rare or difficult to culture pathogen (e.g., the TB bacillus) can also be determined by probing gDNA with a hybridization probe specific to a gene possessed by the pathogen. Similarly, the heterozygous presence of a mutant allele in a phenotypically normal individual, or its homozygous presence in a fetus, may be determined by the utilization of an allele-specific probe that is complementary only to the mutant allele. See e.g., Guo, N. C. et al. (1994) Nucleic Acid Research 22:5456-5465).

Currently, all of the existing methodologies which utilize single-gene probes, if applied to determine all of the genes expressed within a given tissue sample, would require many thousands to tens-of-thousands of individual probes. It has been estimated that a single human cell typically expresses approximately 5,000 to 15,000 genes simultaneously, and that the most complex types of tissues (e.g., brain tissue) can express up to one-half of the total genes contained within the human genome. See Liang, et al. (1992) Differential Display of Eukaryotic Messenger RNA by Means of the Polymerase Chain Reaction, Science 257:967-971. It is obvious that a screening methodology, which requires such a large number of probes, is clearly far too cumbersome to be economic or, even practical.

In contrast, another class of existing methods, known as sequencing-by-hybridization (“SBH”), utilizes combinatorial probes, which are not gene specific. See e.g., Drmanac, et al. (1993) Science 260:1649-1652; U.S. Pat. No. 5,202,231 to Drmanac, et al. An exemplar implementation of SBH for the determination of an unknown gene requires that a single cDNA clone be probed with all DNA oligomers of a given length, say, for example, all 6 nt oligomers. A set of oligomers of a given length, which are synthesized without any type of selection, is called a combinatorial probe library. A partial DNA sequence for the cDNA clone can be reconstructed by algorithmic manipulations from the hybridization results for a given combinatorial library (ie., the hybridization results for the 4096 oligomer probes having a length of 6 nt). However, complete nucleotide sequences are not determinable, because the repeated subsequences cannot be fully ascertained in a quantitative manner.

SBH, which is adapted to the identification of known genes, is called oligomer sequence signatures (“OSS”). See e.g., Lennon, et al. (1991) Trends In Genetics 7(10:314-317. OSS classifies a single clone based upon the pattern of probe “hits” (ie., hybridizations) against an entire combinatorial library, or a significant sub-library. This methodology requires that the tissue sample library be arrayed into clones, wherein each clone comprises only a single sequence from the library. This technique cannot be applied to mixtures of sequences.

These previous, exemplar methodologies are all directed to finding one sequence in an array of clones—with each clone expressing a single sequence from a given tissue sample. Accordingly, they are not directed to rapid, economical, quantitative, and precise characterization of all the DNA sequences in a mixture of sequences, such as a particular total cellular cDNA or gDNA sample, and their adaptation to such a task would be prohibitive. Determination by sequencing the DNA of a clone, much less an entire sample of thousands of genomic sequences, is not rapid or inexpensive enough for economical and useful diagnostics. As previously discussed, existing probe-based techniques of gene determination or classification, whether the genes are known or unknown, require many thousands of probes, each specific to one possible gene to be observed, or at least thousands or even tens of thousands of probes in a combinatorial library. Further, all of these aforementioned methods require the sample be arrayed into clones each expressing a single gene of the sample.

In contrast to the prior exemplar gene determination and classification techniques, another methodology, known as differential display, attempts to “fingerprint” a mixture of expressed genes, as is found in a pooled cDNA library. This “fingerprint,” however, seeks merely to establish whether two samples are the same or different. No attempt is made to determine the quantitative, or even qualitative, expression of particular genes. See e.g., Liang, et al. (1995) Curr. Opin. Immunol. 7:274-280; Liang, et al. (1992) Science 257:967-971; Welsh, et al. (1992) Nuc. Acid Res. 20:4965-4970; McClelland, et al. (1993) Exs. 67:103-115; and Lisitsyn, (1993) Science 259:946-950. Differential display uses the polymerase chain reaction (“PCR”) to amplify DNA subsequences of various lengths, which are then defined by their being between the annealing sites of arbitrarily selected primers. Polymerase chain reaction method and apparatus are well known. See, e.g., U.S. Pat. Nos. 4,683,202; 4,683,195; 4,965,188; 5,333,675; each herein fully incorporated by reference. Ideally, the pattern of the lengths observed is characteristic of the specific tissue from which the library was originally prepared. Typically, one of the primers utilized in differential display is oligo(dT) and the other is one or more arbitrary oligonucleotides, which are designed to hybridize within a few hundred base pairs (bp) of the homopolymeric poly-dA tail of a cDNA within the library. Thereby, upon electrophoretic separation, the amplified fragments of lengths up to a few hundred base pairs should generate bands that are characteristic and distinctive of the sample. In addition, changes in gene expression within the tissue may be observed as changes in one or more of the cDNA bands.

In the differential expression methodology, although characteristic electrophoretic banding patterns develop, no attempt is made to quantitatively “link” these patterns to the expression of particular genes. Similarly, the second arbitrary primer also cannot be traced to a particular gene due to the following reasons. First, the PCR process is less than ideally specific. One to several base pair mismatches are permitted by the lower stringency annealing step that is typically utilized in this methodology and are generally tolerated well enough so that a new chain can actually be initiated by the Tag polymerase often used in PCR reactions. Secondly, the location of a single subsequence (or its absence) is insufficient to distinguish all expressed genes. Third, the resultant bp-length information (ie., from the arbitrary primer to the poly-dA tail) is generally not found to be characteristic of a sequence due to: (i) variations in the processing of the 3′-untranslated regions of genes, (ii) variation in the poly-adenylation process and (iii) variability in priming to the repetitive sequence at a precise point. Therefore, even the bands that are produced often are smeared by numerous, non-specific background sequences.

Moreover, known PCR biases towards nucleic acid sequences containing high G+C content and short sequences, further limit the specificity of this methodology. In accord, this technique is generally limited to the “fingerprinting” of samples for a similarity or dissimilarity determination and is precluded from use in quantitative determination of the differential expression of identifiable genes.

Thus, in conclusion, the existing methodologies utilized for gene or DNA sequence classification and determination are in need of improvement with respect to their ability to perform a highly specific quantitative determination of the components of a cDNA mixture prepared from a tissue sample in a rapid, economical and reproducible manner.

### SUMMARY OF THE INVENTION

The invention provides methods of characterizing a polynucleotide sequence. The polynucleotide is for example, genomic DNA, cDNA or alternatively RNA. No particular length is implied by nucleotide sequence. Any length polynucleotide sequence is characterized by the methods of the invention. In one aspect, the method includes providing a linear nucleic acid sequence of known length and with a defined 5′ terminus and a defined 3′ terminus. The 5′ and 3′ termini can be determined by methods known in the art including hybridization and sequencing. In a preferred embodiment, restriction endonuclease cleavage sites define the 5′ and 3′ termini. By defined it is meant that the identity of the nucleotides at the termini are known. Preferably, at least 2, 3, 4, 5, 6, 7, 8, 9 or more terminal nucleotides are known. The linear nucleic acid sequence is contacted with a restriction endonuclease and whether the restriction endonuclease cleaves the linear nucleic acid sequence is determined, thereby characterizing the polynucleotide sequence.

In some embodiments, the 5′ terminus and 3′ terminus have the same restriction endonuclease cleavage sites defining them. In alternative embodiments, different restriction endonuclease cleavage sites define the 5′ terminus and 3′ terminus. The restriction endonucleases recognize a sequence of at least four nucleotides. Alternatively, restriction endonucleases recognize a sequence of at least six nucleotides.

In further embodiments, the linear nucleic acid sequences are contacted with an additional restriction enzyme. For example, the nucleic acid sequence is contacted with at least 2, 3, 4, 5, 6, 7, 8, 9, 10, 11 or more restriction enzymes.

The invention further provides methods of identifying a polynucleotide sequence by providing information for a first linear nucleic acid sequence or fragment. The information includes for example, the length of the first linear nucleic acid sequence, a defined 5′ terminus and a defined 3′ terminus, and the cleavage status for at least one additional restriction endonuclease. The 5′ and 3′ termini are restriction endonuclease cleavage sites. As used herein, the term “cleavage status” refers to whether a nucleic acid fragment is cut by a restriction enzyme or remains uncut. Thus, the cleavage status provides information as to whether a nucleotide subsequence (ie., the restriction endonuclease site) is present or absent in the nucleic acid fragment. The cleavage status can be determined by standard methods known to those of skill in the art such as, gel electrophoresis. The information for first linear nucleic acid sequence is compared to the information for a second linear nucleic sequence. The first polynucleotide sequence is identified where the information for the first linear nucleic acid sequence matches or is similar to the information for the second linear nucleic acid sequence thereby indicating that the first linear nucleic acid sequence is the second linear nucleic acid sequence.

In some embodiments of the invention, the second linear nucleic acid sequence is a member of a plurality of polynucleotide sequences. In other embodiments, the first linear nucleic acid sequence is a member of a plurality of polynucleotide sequences.

The details of one or more embodiments of the invention have been set forth in the accompanying description below. Although any methods and materials similar or equivalent to those described herein can be used in the practice or testing of the present invention, the preferred methods and materials are now described. Other features, objects, and advantages of the invention will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this invention belongs. All patents and publications cited in this specification are incorporated by reference.

### BRIEF DESCRIPTION OF THE FIGURES

FIG. 1 is a schematic representation showing the information obtained from incubating a nucleotide fragment/band with a restriction enzyme.

FIG. 2 is a schematic representation showing the data obtained from clipping illustrating the assignment of a band to a gene.

FIG. 3 is a graph showing the cutting frequency of enzyme AciI for a set of traces.

FIG. 4A is a graph showing the trace of the results of digesting band 112.3 with AluI, BfaI, HaeIII, and HinPI-I.

FIG. 4B is a graph showing the trace of the results of digesting band 112.3 with MseI, RsaI, AciI and MspI.

FIG. 5 is a graph showing the total bands and the number of bands that are assigned to known genes determined by clipping.

### DETAILED DESCRIPTION OF THE INVENTION

The invention is based in part on the discovery of a method for identifying or classifying a polynucleotide sequence. The invention allows for a highly efficient method of assigning a gene identity to a nucleic acid sequence or fragment. Furthermore, the method can be used to quickly classify or identify a population of polynucleotides, for example in determining differential gene expression. The method is referred to herein as clipping. Briefly, the method proceeds by performing a series of restriction enzyme (RE) digestions of nucleic acid fragments of known length and known terminal sequences. The data obtained from the series of digests comprises whether a subsequence corresponding to a restriction endonuclease cleavage site is absent or present within the sequence of interest based upon the cleavage status (ie., whether the fragment remains uncut or is cut by the RE). This allows for a binary pattern to be assigned to each sequence, wherein 0 corresponds to uncut and 1 corresponds to cut. For example, a nucleic acid sequence is identified by comparing the cleavage status of the polynucleotide sequence or fragment to a virtually digested putative sequence identity in order to assign that gene to the sequence.

Without limitation, the clipping methodology disclosed herein has been primarily applied in quantitative expression analysis (GeneCalling™), as disclosed in U.S. Pat. No. 5,871,697 entitled “METHOD AND APPARATUS FOR IDENTIFYING, CLASSIFYING, OR QUANTIFYING DNA SEQUENCES IN A SAMPLE WITHOUT SEQUENCING,” and which is incorporated herein by reference in its entirety. However, individuals possessing ordinary skill within the relevant arts will immediately appreciate how to adapt this methodology to the other protocols for generating appropriate nucleic acids samples of the described structure. Prior to proceeding with the detailed description of the clipping methodology, a review of GeneCalling™ methodology will be set forth.

Quantitative Expression Analysis (GeneCalling™)

In order to uniquely identify or classify an expressed, full or partial nucleotide or gene sequence, as well as many components of genomic DNA (gDNA), it is not necessary to determine the actual, complete nucleotide sequences, as these complete nucleotide sequences provide far more information than is needed to merely classify or determine a given nucleotide sequence according to the present invention disclosed herein. Moreover, the actual number of expressed human genes represents an extremely small fraction (ie., 10⁻¹¹⁹⁵) of the total number of possible DNA sequences. Hence, the utilization of GeneCalling™ and the clipping methodologies of the present invention, allows direct determination of nucleotide sequences (without the requirement of establishing a complete nucleotide sequence) within a heterogeneous sample by making use of a nucleic acid sequence database containing those of sequences that are likely to be present within the sample. Moreover, even if such a database is not available, sequences within the sample can, nonetheless, be individually classified.

Quantitative expression analysis (GeneCalling™) provides a methodology for identifying, classifying, or quantifying one or more nucleic acids sequences within a sample comprising a plurality of nucleic acids species each possessing different nucleotide sequences. In brief, the various steps in the preferred embodiment of GeneCalling™ methodology (ie., the restriction endonuclease digestion/ligation/amplification-based protocol) may be summarized as follows:

Step 1: complementary DNA (cDNA) synthesis

Step 2: The resulting cDNA fragments are digested utilizing two different restriction endonucleases (RE) which, preferably, recognize only rare, 6-8 bp RE-recognition sequences.

Step 3: Ligation of oligonucleotide “adapters” to the digested cDNA fragments. Two different adapters are utilized, with each adapter being complementary to the sequences of one of the two RE recognition sites.

Step 4: PCR amplification is performed utilizing labeled primers that are complementary to the two adapters ligated to the digested cDNA fragments.

Step 5: The reaction products of the PCR amplification are then electrophoresed to observe the electrophoretic mobility patterns of the individual fragments. These mobility patterns are then utilized to construct an electropherogram.

Step 6: From the electrophoretic mobility and electropherogram the sizes of the individual fragments of interest are identified, and a computer DNA sequence database is then searched to generate a list of putative gene “identities” for these aforementioned fragments.

Thus, the GeneCalling™ methodology is performed by hybridizing the sample with one or more labeled probes, wherein each probe recognizes a different “target” nucleotide subsequence or a different set of “target” nucleotide subsequences. The target subsequences utilized in the GeneCalling™ methodology are, preferably, optimally chosen by computer-implemented methods in view of DNA sequence databases containing sequences likely to occur in the sample to be analyzed. In respect to the analysis of human genomic cDNAs, efforts of the Human Genome Project in the United States, efforts abroad, and efforts of private companies in the sequencing of the human genome sequences, both expressed and genetic, are being collected in several available databases.

The resulting hybridization signal(s) is, preferably, comprised of a representation of (i) the presence of a first target subsequence, (ii) the presence of a second target subsequence, and (iii) the length between the target subsequences in the sample nucleic acid sequence. If the first strand of target subsequences occurs more than once in a single nucleic acid in the sample, more than one signal is generated, each signal comprising the length between adjacent occurrences of the target subsequences. While the target subsequences recognized are typically contiguous, the GeneCalling™ methodology is adaptable to recognizing discontiguous target subsequences or discontiguous effective target subsequences. For example, oligonucleotides recognizing discontinuous target subsequences can be constructed by inserting degenerate nucleotides within a discontinuous region. In addition, phasing primers (which possess additional nucleotide sequence beyond the RE site) may also be utilized to augment sequence specificity.

Following hybridization and target signal detection, a search of a nucleotide sequence database, comprised of known sequences of nucleic acids which may be present within the sample, is performed in order to ascertain either sequences which match or, alternately, the absence of any sequences which match the generated hybridization signal(s). A sequence contained within the database is considered to “match” (ie., is homologous) to a generated hybridization signal when the nucleotide sequence from the database possesses both (i) the same length between occurrences of the target subsequences as is represented by the generated hybridization signal and (ii) the same target subsequences as is represented by the generated signal or, alternately, target subsequences which are members of the same sets of target subsequences represented by the generated signal.

The GeneCalling™ methodology may be applied to the analysis of complementary DNA (cDNA) samples synthesized from any in vivo or in vitro sources of RNA. cDNA can be synthesized from total cellular RNA, poly(A)′ messenger RNA (mRNA), or from specific sub-pools of RNA. RNA pre-purification can produce such RNA sub-pools. For example, the separation of endoplasmic reticulum mRNA species from those mRNAs contained within the cytoplasmic fraction facilitates the enrichment of mRNA species that encode cell surface or extracellular proteins. See e.g., Celis, L., et al., 1994. Cell Biology (Academic Press, New York, N.Y.).

While the GeneCalling™ methodology is preferred for classifying and determining sequences contained within a sample comprised of a mixture of cDNAs, it is also adaptable to samples that contain a single cDNA moiety. Typically, enough pairs of target subsequences can be chosen so that sufficient distinguishable signals may be generated so as to allow the determination of one, to all of the sequences contained within the sample mixture. For example, in a first possible scenario, any pair of target subsequences may occur more than once in a single DNA molecule to be analyzed, thereby generating several signals with differing lengths from one DNA molecule. In a second scenario, even if a pair of target subsequences occurred only once within two different DNA molecules to be analyzed, the lengths between the probe hybridizations may differ, and thus distinguishable hybridization signals may be generated.

In the preferred PCR-mediated GeneCalling™ methodology, a suitable collection of target subsequences is chosen via computer-implemented methods and PCR primers, preferably labeled with fluorescent moieties, are synthesized to hybridize with these aforementioned target subsequences. Advances in fluorescent labeling techniques, in optics, and in optical sensing currently permit multiply-labeled DNA fragments to be differentiated, even if they spatially-overlap (ie., occupy the same “spot” on a hybridization membrane or a band within a gel). See Ju, T., et al., 1995. Proc. Natl. Acad Sci. USA 92:4347-4351. Accordingly, the results of several GeneCalling™ reactions may be multiplexed within the same gel lane or filter spot. The primers are designed to reliably recognize short subsequences while achieving a high specificity in the PCR amplification step. Utilizing these primers, a minimum number of PCR amplification steps amplify those fragments between the primed subsequences existing in DNA sequences in the sample, thereby recognizing the target subsequences. The labeled, amplified fragments are then separated by gel electrophoresis and detected.

GeneCalling™ may be performed in either a “query mode” or in a “tissue mode.” In query mode, the focus is upon the determination of the expression of a limited number of genes of interest and of known sequence (e.g., those genes which encode oncogenes, cytokines, and the like). A minimal number of target subsequences are chosen to generate signals, with the goal that each of the limited number of genes is discriminated from all the other genes likely to occur in the sample by at least one unique signal. Conversely, in tissue mode, the focus is upon the determination of the expression of as many as possible of the genes expressed in a tissue or other sample, without the need for any prior knowledge of their expression. In the tissue mode, target subsequences are optimally chosen to discriminate the maximum number of sample DNA sequences into classes comprising one, or preferably at-most a few sequences. Ideally, sufficient hybridization signals are generated and detected so that computer-based identification methods can uniquely determine the expression of a majority, or more preferably most, of the genes expressed within a given tissue. It should be noted, however, that in both modes, hybridization signals are generated and detected as determined by the threshold and sensitivity of a particular experiment. Important determinants of threshold and sensitivity include, but are not limited to: (i) the initial amount of mRNA and thus of cDNA utilized; (ii) the amount of PCR-mediated amplification performed and (iii) the overall sensitivity and discrimination capability of the detection means utilized.

Clipping Methodology

Without limitation, the following description of the clipping methodology has been primarily directed towards application to GeneCalling™. However, an individual of ordinary skill within the relevant arts will immediately appreciate how to adapt this description to other protocols for generating appropriate nucleic acids samples of the described structure.

Clipping is applicable for nucleic acid fragments of any size which possess the ability to be cut by restriction enzymes (REs), including, but not limited to, those nucleic acid fragments typically present within GeneCalling™ reaction products which generally range in size from 30-600 bp in length. Typically, the clipping methodology proceeds by performing a series of RE digestions of GeneCalling™ reaction products, which is designed to produce detectable results for those amplification products that do not possess a uniquely identified sequence. In the preferred embodiment, this result is achieved by incubating the GeneCalling™ products with a series of REs. The results of the digests show whether each restriction enzyme's cleavage site is present or absent as an additional subsequence. However, clipping is also equally applicable to identifying, narrowing, or confirming putative sequence identifications in any sample of nucleic acid fragments which possess a defined “generic” structure or motif which will be discussed supra. The only imposed limitation is that these nucleic acid fragments must possess known terminal subsequences, ie., the 5′ terminus and the 3′ terminus are defined. Several methodologies for producing nucleic acids with such a generic structural motif are well known to those individuals skilled in the art.

The aforementioned generic structural motif is comprised of nucleic acid species possessing known or defined terminal subsequences on both the 3′- and 5′-termini, which flank a central subsequence of interest. The terminal subsequences may be different and of any length. A minimum length of at least two base pairs of known or defined sequence at both the 3′ terminus and the 5′ terminus is preferred. While the central subsequence may be of any length, a minimum length of approximately 10 bp is preferred in the present invention. The central subsequence determines the “identity” of the specific nucleic acid species, and is thereby to be compared with the putatively identified sequence. Hence, confirmation is obtained if a fragment exists within the sample, which possesses a central subsequence having a sequence that is (at a minimum) homologous to a portion of the putatively identified sequence.

Nucleic acids possessing this generic structural motif are, preferably, produced according to the GeneCalling™ methods of this invention. A preferred embodiment of the clipping methodology is utilized in confirming that a specific sequence, obtained through the use of a nucleic acid sequence computer database, which has been predicted to generate a particular GeneCalling™ signal is, in actuality, generating the signal. Nonetheless, this embodiment of the clipping methodology is not limited to confirming the results of the GeneCalling™ methodology, and can be equally applied to assigning a gene identity to or confirming the results obtained from any other protocol utilizing nucleic acid species possessing the previously described generic structural motif. Therefore, as will be apparent to those of skill in the art, the clipping methodology may be, more generally, utilized to assign or confirm a putative sequence identification of a fragment within a sample of nucleic acid fragments possessing the aforementioned generic structural motif.

Several methods have been described in the art for the generation of such nucleic acid species from biological nucleic acid samples, however Applicants do not hereby admit that any of the subsequently described examples contained herein are prior art to their invention. Three such exemplar methodologies will now be briefly described. A first method is disclosed in European Patent Application 0 534 858 A1, entitled “Selective Restriction Fragment Amplification: A General Method for DNA Fingerprinting,” and which is incorporated by reference herein in its entirety. According to this method, a sample of cDNA is initially digested with restriction endonucleases (“RE”) into fragments and oligonucleotides complementary to these digested fragments are hybridized to the fragments. A longer primer strand of each adaptor is then ligated to the fragments. These products are then PCR amplified using PCR primers that include the longer primer strands. For selective amplification, these primers can, optionally, extend for 1-10 selected nucleotides beyond any remaining portion of the RE recognition site. Since fragments in the unamplified, amplified, and selectively amplified samples are all terminated by known primer sequences, this method generates nucleic acid samples of the described generic structure. In accord with this method, partial or complete sequencing can putatively identify the sequences of individual fragments within these samples.

A second method is described in U.S. Pat. No. 5,459,037, entitled “Method for Simultaneous Identification of Differentially Expressed mRNAs and Measurement of Relative Concentrations,” which is incorporated by reference herein in its entirety. As disclosed by this method, cDNAs are synthesized using a first-strand oligo(dT) primer, which includes two phasing nucleotides and a recognition site for a rare-cutting RE. The resulting cDNAs are then digested by both the rare-cutting RE and a more frequently-cutting RE. The digested fragments are ligated in an anti-sense orientation into a cloning vector, which is subsequently used to synthesize complementary RNA (cRNA). Next, cDNA is synthesized from this cRNA using first-strand primers having sequences corresponding to the portion of the cloning vector adjacent to the 3′-termini of each insert, as well as including two phasing nucleotides. Finally, the resulting products are PCR amplified using primers comprising adjacent portions of the cloning vectors on both sides of the insert, with one of these primers having optional phasing nucleotides. Since nucleic acid fragments in all the multiple, possible pools of final samples are terminated by known primer sequences, this methodology generates nucleic acid species of the previously described generic structural motif. According to this method, partial or complete sequencing can putatively identify the sequences of individual fragments in these samples.

A third method is described in Prashar, et al., 1996. Analysis of Differential Gene Expression by Display of 3′-End Restriction Fragments of cDNAs, Proc. Nat. Acad. Sci. USA 93:659-663, which is incorporated by reference herein in its entirety. As disclosed by this method, cDNA is synthesized using an oligo(dT) first-strand primer possessing two phasing nucleotides at the 3′-terminus and a special “heel” subsequence at the 5′-terminus. After digestion with a frequently-cutting RE, a partially double-stranded “Y”-adapter is annealed and ligated onto the RE-digested termini of the cDNA fragments. This “Y”-adaptor possesses a non-complementary region including a 5′-primer sequence. Finally, PCR amplification of the ligated fragments, which are primed with a first primer having the heel primer sequence and a second primer having the 5′-end primer sequence, produces a pool of fragments that have been terminated by these aforementioned sequences. Similarly, since the pool of final fragments is terminated by known primer sequences, this method generates nucleic acid species of the previously described generic structural motif. According to this method, partial or complete sequencing can putatively identify the sequences of individual fragments in these samples.

As previously discussed, clipping is also adaptable to other methodologies which utilize nucleic acid fragment samples having the aforementioned generic structural motif which are either known within the art, or subsequently described in the future. As confirmatory oligo-poisoning methodologies are, preferably, applied to GeneCalling™ reaction products, they are described in the following subsection primarily with respect to such GeneCalling™ reaction products. However, this description is without limitation, as individuals possessing ordinary skill within the relevant arts will readily appreciate how to adapt clipping methodologies to any sample of nucleic acids which possess the previously-described generic structural motif, including nucleic acid species produced by the aforementioned methods and the like.

Confirmation of a Putative Sequence by the Clipping Methodology

The clipping methodology disclosed herein may be utilized to confirm a putative sequence that has been identified for a nucleic acid fragment, within a sample of nucleic acids, possessing the previously described generic structural motif. The clipping methodology depends upon the knowledge of, and serves to confirm the nucleotide sequence of, a portion of a unique, central nucleic acid sequence of interest, which is spatially located adjacent to known terminal subsequences. It has been ascertained that the knowledge of (at a minimum) the sequence of a portion of a fragment is, in fact, sufficient to confirm that a putative, candidate sequence, or which one of a small number of putative, candidate sequences, is actually the sequence of the nucleic acid species of interest.

The confirmation of the GeneCalling™ sequence identification for a nucleic acid of interest utilizing the clipping methodology is, preferably, performed in the following manner. An aliquot of the nucleic acid sample is incubated with a restriction enzyme. The digested aliquot is then separated, preferably, via gel electrophoresis, and the resultant separated bands are detected and analyzed in an appropriate manner (ie., automated optical detection with the generation of an electropherogram). The results of the clipping RE digest reaction are compared with those results obtained from virtual digestion of the putative gene identities with the same enzyme.

Accordingly, if the nucleic acid fragment of interest possesses a correctly identified putative sequence, it will match the binary pattern obtained by the virtual digestion of the identified putative sequence.

In brief, the clipping methodology, as applied to GeneCalling™ confirmation, is comprised of the following steps:

Step 1: A PCR amplified GeneCalling™ reaction is performed as described in U.S. Pat. No. 5,871,697.

Step 2: Utilizing the electrophoretic mobility results obtained from the electrophoresis of the GeneCalling™ PCR amplification reaction products in combination with those putative sequence “identity” results obtained from the utilization of the nucleic acid sequence database, a set of REs is chosen to “clip” the amplification products.

Step 3: A series of RE digestions are performed using buffer and incubation conditions appropriate for each enzyme.

Step 4: The reaction products of the clipping digests are electrophoresed to observe the electrophoretic mobility patterns of the individual fragments and an electropherogram is constructed.

Step 5: A binary pattern is produced based on whether the fragment is uncut (=0) or cut (=1).

Step 6: The putative gene identity or identities are subjected to virtual digestions with the same enzymes to generate a binary pattern, which is compared to the experimental binary pattern for the nucleic acid fragment.

The clipping methodology can also advantageously be applied to nucleic acid fragments of interest in each of two or more samples of nucleic acids that possess the previously described generic structural motif. Such samples may be obtained, for example, from two or more comparable tissue samples which are in different biological “states.” In the aforementioned case, clipping may be utilized to confirm the putative identification of fragments having expression differences between the samples (ie., exhibiting differential expression), and to determine whether a novel nucleic acid is generating such expression differences.

For example, in the case of a fragment of interest which has been determined to be differentially expressed in each of two tissue samples (e.g., by a previous electrophoretic comparison) and which has been identified as possibly possessing two or more putative candidate sequences, the sequential digestion of the fragments with REs may be utilized to identify the differential and relative presence of each candidate sequence within each tissue. In one potential scenario, the expression of both candidate sequences may be differentially increased within the same tissue sample, thus leading to a greater differential expression of the fragment of interest between the two tissues. In a second potential scenario, the expression of the candidate sequences may be differentially increased within different tissue samples, leading to a lesser differential of the fragment of interest. The clipping methodology possesses the ability to ascertain which of these potential scenarios is correct.

Preferred Clipping Methodology

GeneCalling® is a differential display method for measuring gene expression levels. This method uses restriction enzyme pairs to cut cDNA pool. In general, tissues are removed and total RNA is prepared from them. cDNA is prepared and the resulting samples are processed using up to 140 subsequences originating, for example, from the recognition sequences of restriction endonucleases. The digested fragments are ligated with complementary adapters and then amplified by PCR using fluorescence labeled primers. The fragments are gel electrophoresed and detected by laser excitation. The genes responsible for the fragments are found by comparing experimentally detected bands to a database of bands predicted for known gene sequences.

Sample preparation and GeneCalling® analysis are described fully in U.S. Pat. No. 5,871,697 and in Shimkets et al., “Gene expression analysis by transcript profiling coupled to a gene database query” Nature Biotechnology 17:198-803 (1999), incorporated herein by reference in their entireties.

The disadvantage of fragment-to-gene database look-up is that, depending on the complexity of the cDNA pool, multiple genes (from a few to a few hundreds) could generate a particular fragment. Therefore, a detected fragment cannot be unambiguously assigned to one gene. It is very inefficient to use trial-and-error to narrow the list of putative gene candidates to the one gene that generates a particular fragment. There are methods like clone sizing and trapping that are used to increase the GeneCalling® resolution. This application is directed to a new method to improve GeneCalling® resolution and the use of this method in finding polymorphisms.

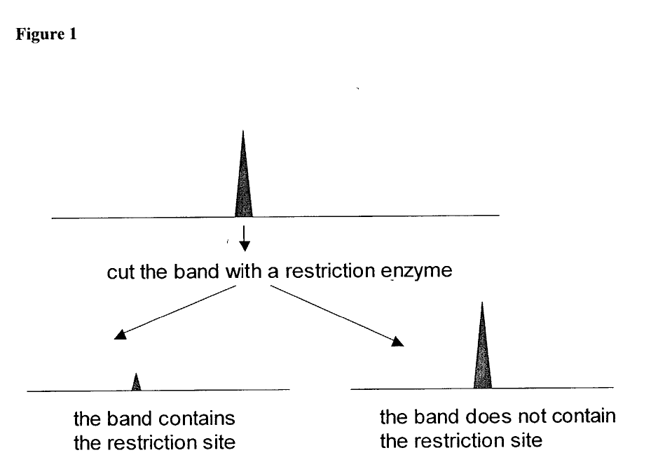

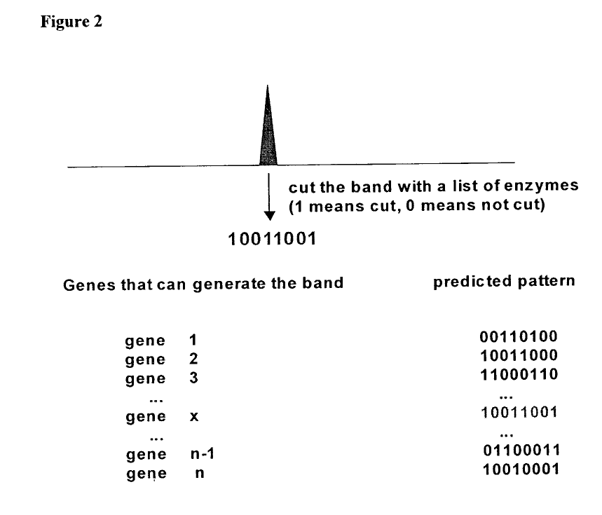

For each fragment after electrophoresis its length and the restriction enzyme pair that was used to generate it are the known parameters. The latter information provides nucleotide subsequences at each end of the fragment determined by the recognition sequences of the respective enzymes. Often, this information is not enough to assign this band to a unique gene. To get more information needed for assigning each band to a unique gene, the band is incubated with an additional set of restriction enzymes. Each enzyme will either cut the fragment or not depending on whether that fragment contains the site recognized by the restriction enzyme (see FIG. 1). Thus each enzyme digestion will generate a bit of information for that band. Using a set of enzymes, a binary pattern is generated for each band (e.g., 10001001, where 0=uncut and 1=cut). Then for each gene that could generate this fragment, a theoretical digestion is performed that generates a predicted binary pattern for that gene (see FIG. 2). By comparing the detected pattern and the predicted pattern the fragment is assigned to only one gene or to a very short list of genes depending on the number of enzymes chosen.

As used herein, the term “clipping”, and similar terms, relate to a procedure whereby a nucleic acid fragment characterized by a first subsequence, a second subsequence, and the distance, or length, in numbers of nucleotides between them, is further characterized. Specifically, this additional characterization relates to the presence or absence of at least one additional nucleotide subsequence in the fragment. This is illustrated schematically in FIG. 1. If a band contains the restriction site, it will disappear after incubation with the enzyme because the enzyme cuts it. If a band does not contain the restriction site, it will remain unchanged after incubation because the enzyme does not cut it. FIG. 2 is a schematic representation depicting the use of clipping to assign a band to a gene. The band was digested with a set of enzymes and generated a pattern 10011001 (1 means cut, 0 means not cut). This band can be assigned to a list of genes (gene 1 to gene n) by database look-up. Each of the genes will generate a pattern by virtual digestion with the same enzymes used above. By comparing the predicted pattern with the detected pattern this band is unambiguously assigned to gene x.

Efficiency

In GeneCalling® a band is assigned to a long list of genes by a database look-up. Each predicted gene contains the two restriction sites and generates a fragment with a length approximately that of the experiment band. If an enzyme will cut half of the predicted fragments generated from that long list of genes, then depending on whether that band disappears or not from the experiment, that band can be assigned to only half of the original list. If a second enzyme can also cut half of the predicted fragments, the list is shortened half again. If the band is digested with n enzymes, the final gene list will be ½ⁿ of the original list, ie. GeneCalling® resolution is improved 2ⁿ times.

Longer fragments are more frequently cut than the shorter fragments. Therefore, if the cutting frequency of the enzymes is not 50%, the efficiency will be less than 2. Although the improvement to GeneCalling™ resolution is depends on the cutting frequency of each enzyme chosen, the resolution can be increased 2, 4, 8, 16, 32, 64, 128 or more times that of GeneCalling™ alone.

Application of Clipping to Finding Polymorphisms

Clipping can be used to find nucleotide polymorphisms in genes. For example, incubating DNA fragments with 12 restriction enzymes will produce 12 bits of information for each fragment. A computer-aided database search may identify a fragment from a gene that contains the same 12 bits. In that case, the band is assigned to this gene.

There are three potential reasons for failure to identify a match. First, there could be an experiment error that caused no match when a match actually exists. Second, the fragment originates from a novel gene. Third, there is a polymorphism in that fragment which leads to a deletion of or an addition of a restriction site. In the case of a nucleotide polymorphism, 11 of the 12 bits of information will match between the experimental fragment digestions and the theoretical database digestions. If 12 enzymes are used and most of them are 4-cutters, the chance of finding a polymorphism in that fragment is high. If there is a polymorphism, the polymorphism position can be located based on the position of the restriction site.

### EXAMPLES

### Example 1

### Computer Simulation

A computer simulation was performed in order to find a list of enzymes that will give a high efficiency for clipping. As discussed previously, the ideal enzymes should have a cutting frequency of 50%. The electrophoretic bands are in the range of 35 to 450 nucleotides long. Since the possibility of a band being cut by an enzyme depends on the length of that band, the longer the band, the more chance an enzyme will cut it. The bands on a representative trace were divided into four ranges (35-135, 135-235, 235-335, 335-450). For each range, all of the genes that can generate the bands in that range are ascertained by a database look-up. Then, a virtual digestion is performed on the corresponding bands from the candidate genes for a set of enzymes. Thus, the cutting frequency of each enzyme in each range can be calculated.

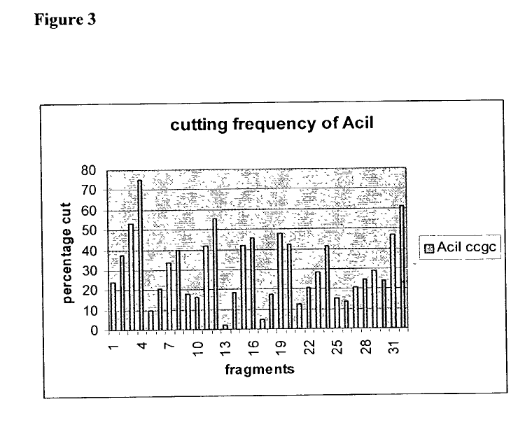

FIG. 3 is a graph of a computer simulation of cutting frequency by enzyme AciI. The x-axis shows the bands in a certain range and generated by a certain pair of restriction enzymes. Lines 1-4 were generated by enzyme pair b1i0, 5-8 by d0p0, 9-12 by g0m0, 13-16 by h0n0, 17-20 by i0n0, 21-24 by m0r0, 25-28 by s0g1, 29-32 by u0f0. Within each group the fragments are divided into four regions, 35-135, 135-235, 235-335, 335-450. Line 1 contains all fragments digested by b1i0 and in the range of 35-135 nts, line 2 contains all fragments in the range of 235-335, line 3 contains all fragments in the range of 235-335, line 4 contains all fragments in the range of 335-450. The database used was GeneBank Rat.

The computer simulation results showed that most 4-cutters have an overall cutting frequency of 50%. In contrast, the most frequent 6-cutters were found to have a cutting frequency of 10-20%. Therefore 4-cutters are preferred for clipping.

### Example 2

### Experimental Design for Clipping

Samples of DNA resulting from GeneCalling® Chemistry were pooled. 1 μl of a 1/1000 dilution of the pooled product was then combined with 9 μl of a digest mix composed of 5-20 units of the restriction endonuclease AciI, AluI, HpyCH4V, HaeIII, HinPI-I, MnlI, MseI, RsaI, NIaIII, MspI, HpyCH4IV, BfaI, or MboI with appropriate buffer and BSA if required. This mixture was incubated at 37 C for 4 hours. 2 ul of the digested material was amplified by PCR in a 50 μl reaction as described previously, but using 24.8 μM of carrier primer. The PCR profile was 95C for 5 min, (95C for 30 sec., 57C for 1 min., 72C for 2 min.) for 20 cycles.

### Example 3

### Data Analysis/Results

For each sample digested with a pair of restriction enzymes a “normal trace” is generated, ie., an electrophoresis of fragments generated from digestion with this pair of enzymes. After further digestion with a series of third restriction enzymes, another set of traces is generated, each corresponding to a digestion with a third enzyme. The traces with and without a third enzyme digestion are compared, thereby determining whether each band is cut or not cut. The result is a binary pattern for each band. This experimentally obtained binary pattern is then compared to the pattern generated by theoretical digestion of the predicted bands in order to assign bands to the genes that generate the same pattern.

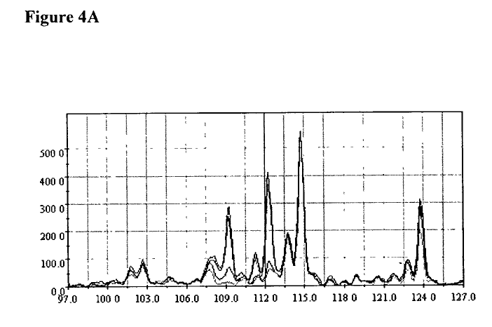

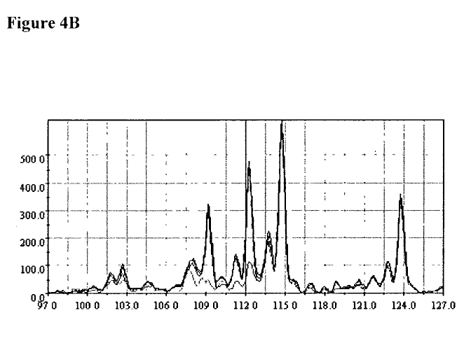

Four subsequence pairs (b1i0, d0p0, h0n0, m0r0) were randomly chosen to run clipping on a rat liver DNA sample. From the computer simulation result, 8 enzymes were chosen to incubate with subsequence d0p0. Those enzymes are AluI, BfaI, HaeIII, HinPI-I, MseI, RsaI, AciI and MspI. Band 112.3 is shown here because this band was confirmed to a gene by poisoning. The eight traces corresponding to the digestions by the eight enzymes are displayed in FIGS. 4A and 4B. The 4 traces shown in FIG. 4A were incubated with AluI, BfaI, HaeIII, and HinPI-I. The 4 traces shown in FIG. 4B were incubated with MseI, RsaI, AciI and MspI. Band 112.3 disappeared after digestion with AluI, BfaI and RsaI.

Thus, the binary pattern from incubations with these 8 enzymes for d0p0 112.3 is 11000100. Then the GeneBank Rat database was searched and all sequences that could generate band d0p0 112.3 were retrieved. A theoretical digest with each of the 8 enzymes is used to obtain a binary pattern representing the cleavage status of each retrieved sequence. Table 1 lists the Accession Number and digestion pattern (ie., binary pattern) of each retrieved sequence that could generate d0p0 112.3

TABLE 1
Theoretical Digestion Patterns for d0p0112.3 Gene Candidates

| Accno | AluI | BfaI | HaeIII | HinPI-I | MseI | RsaI | AciI | MsPI |
|---|---|---|---|---|---|---|---|---|
| ab010436 | 1 | 0 | 0 | 0 | 0 | 0 | 1 | 1 |
| ab010437 | 1 | 0 | 0 | 0 | 0 | 0 | 1 | 1 |
| ab012279 | 1 | 0 | 0 | 0 | 0 | 0 | 0 | 0 |
| ab022882 | 0 | 1 | 0 | 0 | 0 | 1 | 0 | 0 |
| ab024398 | 1 | 1 | 0 | 0 | 1 | 0 | 0 | 0 |
| ab024400 | 0 | 0 | 1 | 0 | 1 | 0 | 0 | 0 |
| ab032243 | 1 | 0 | 0 | 0 | 0 | 0 | 0 | 0 |
| ab032419 | 0 | 0 | 1 | 0 | 0 | 0 | 0 | 1 |
| af019624 | 0 | 0 | 0 | 0 | 0 | 1 | 0 | 0 |
| af021137 | 0 | 0 | 0 | 0 | 0 | 1 | 0 | 0 |
| af031879 | 0 | 0 | 0 | 0 | 0 | 0 | 0 | 0 |
| af069782 | 0 | 0 | 1 | 0 | 1 | 0 | 0 | 0 |
| af076183 | 1 | 0 | 0 | 0 | 1 | 0 | 0 | 1 |
| af076184 | 1 | 0 | 0 | 0 | 1 | 0 | 0 | 1 |
| af087431 | 1 | 0 | 0 | 0 | 0 | 0 | 0 | 0 |
| af102854 | 1 | 0 | 0 | 0 | 0 | 0 | 0 | 0 |
| af142778 | 0 | 0 | 0 | 0 | 0 | 1 | 0 | 1 |
| af194371 | 0 | 0 | 1 | 0 | 1 | 0 | 0 | 0 |
| af234635 | 0 | 1 | 0 | 0 | 0 | 1 | 0 | 0 |
| af239045 | 0 | 1 | 0 | 0 | 1 | 0 | 0 | 0 |
| af268593 | 1 | 0 | 0 | 0 | 0 | 0 | 0 | 1 |
| af304429 | 0 | 0 | 0 | 0 | 1 | 0 | 0 | 0 |
| aj000696 | 0 | 0 | 0 | 0 | 0 | 1 | 1 | 0 |
| aj001713 | 0 | 0 | 0 | 0 | 1 | 0 | 0 | 0 |
| aj006070 | 0 | 1 | 0 | 0 | 0 | 0 | 0 | 0 |
| d13623 | 1 | 0 | 1 | 0 | 1 | 0 | 0 | 0 |
| d14478 | 0 | 0 | 0 | 0 | 0 | 0 | 0 | 0 |
| d14479 | 1 | 0 | 1 | 0 | 0 | 0 | 0 | 0 |
| d14479 | 0 | 0 | 0 | 0 | 0 | 0 | 0 | 0 |
| d14480 | 1 | 0 | 1 | 0 | 0 | 0 | 0 | 0 |
| d17521 | 0 | 0 | 0 | 0 | 0 | 1 | 0 | 1 |
| d38448 | 1 | 0 | 0 | 0 | 0 | 1 | 0 | 0 |
| d82069 | 1 | 0 | 0 | 0 | 0 | 1 | 0 | 0 |
| d87248 | 1 | 0 | 0 | 0 | 1 | 0 | 0 | 0 |
| d90164 | 1 | 1 | 0 | 0 | 1 | 0 | 0 | 0 |
| k01934_1 | 0 | 0 | 0 | 0 | 1 | 0 | 0 | 0 |
| l15354 | 0 | 1 | 0 | 0 | 0 | 1 | 0 | 0 |
| l15355 | 0 | 1 | 0 | 0 | 0 | 1 | 0 | 0 |
| l19694 | 1 | 0 | 0 | 0 | 1 | 0 | 0 | 0 |
| l21698 | 1 | 0 | 0 | 0 | 0 | 0 | 0 | 0 |
| l21698 | 0 | 0 | 1 | 0 | 0 | 0 | 0 | 1 |
| l46874 | 0 | 0 | 0 | 0 | 1 | 0 | 0 | 0 |
| l48490 | 0 | 0 | 0 | 0 | 0 | 1 | 0 | 0 |
| l48619 | 1 | 1 | 0 | 0 | 0 | 0 | 0 | 0 |
| m17960 | 0 | 0 | 1 | 0 | 0 | 0 | 1 | 0 |
| m21964 | 0 | 0 | 0 | 0 | 0 | 0 | 0 | 0 |
| m29014 | 0 | 0 | 1 | 0 | 1 | 0 | 1 | 1 |
| 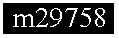 | 1 | 1 | 0 | 0 | 0 | 1 | 0 | 0 |
| m37227 | 0 | 0 | 0 | 0 | 0 | 0 | 0 | 0 |
| m55250 | 0 | 0 | 0 | 0 | 0 | 0 | 0 | 1 |
| m57405 | 0 | 0 | 0 | 0 | 0 | 0 | 0 | 0 |
| m63894 | 0 | 0 | 0 | 0 | 0 | 1 | 1 | 0 |
| m64274 | 1 | 0 | 0 | 0 | 0 | 1 | 0 | 0 |
| s66862 | 1 | 0 | 0 | 0 | 0 | 0 | 0 | 0 |
| s66862 | 0 | 0 | 1 | 0 | 0 | 0 | 0 | 1 |
| s78218 | 1 | 1 | 0 | 0 | 1 | 0 | 0 | 0 |
| u07683 | 1 | 0 | 0 | 0 | 0 | 0 | 0 | 0 |
| u07683 | 0 | 0 | 1 | 0 | 0 | 0 | 0 | 1 |
| u22830 | 0 | 0 | 0 | 0 | 0 | 0 | 0 | 1 |
| u33472 | 0 | 0 | 1 | 0 | 1 | 1 | 0 | 1 |
| u35775 | 0 | 0 | 0 | 0 | 0 | 1 | 0 | 0 |
| u39207 | 0 | 0 | 0 | 0 | 1 | 0 | 0 | 0 |
| u42975 | 1 | 1 | 0 | 0 | 0 | 0 | 0 | 0 |
| u54632 | 1 | 0 | 1 | 0 | 0 | 0 | 0 | 0 |
| u60835 | 0 | 0 | 0 | 0 | 0 | 0 | 0 | 0 |
| u65007 | 1 | 0 | 0 | 0 | 0 | 0 | 0 | 0 |
| u65007 | 0 | 1 | 0 | 0 | 0 | 0 | 0 | 0 |
| u70825 | 1 | 1 | 0 | 0 | 0 | 0 | 1 | 0 |
| u73142 | 1 | 0 | 0 | 0 | 0 | 1 | 0 | 0 |
| u91847 | 1 | 0 | 0 | 0 | 0 | 1 | 0 | 0 |
| x03478 | 1 | 0 | 1 | 0 | 0 | 1 | 0 | 0 |
| x13804 | 0 | 0 | 0 | 0 | 0 | 0 | 0 | 0 |
| x14977 | 0 | 0 | 1 | 0 | 1 | 0 | 1 | 1 |
| x52477 | 0 | 0 | 0 | 0 | 0 | 0 | 0 | 0 |
| x55298 | 0 | 0 | 1 | 0 | 0 | 0 | 0 | 0 |
| 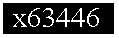 | 1 | 1 | 0 | 0 | 0 | 1 | 0 | 0 |
| x78997 | 0 | 0 | 0 | 0 | 1 | 0 | 0 | 0 |
| x96786 | 1 | 0 | 0 | 0 | 0 | 0 | 0 | 0 |
| x96786 | 0 | 1 | 0 | 0 | 0 | 0 | 0 | 0 |
| x99337 | 0 | 0 | 1 | 0 | 0 | 1 | 0 | 0 |
| x99338 | 0 | 0 | 1 | 0 | 0 | 1 | 0 | 0 |
| z46882 | 1 | 0 | 0 | 1 | 0 | 0 | 0 | 0 |

Of these 82 candidate genes that could generate band d0p0 112.3 only two match the digestion pattern for this band, m29758 and x63446. One of these two, x63446 was 5 confirmed by poisoning (see Table 2). Poisoning is a PCR method in which competing primers that carry no label are used. If the unlabeled primer is incorporated, the corresponding band for the amplified fragment should diminish in amplitude, or essentially disappear. The other candidate gene, m29758, was found to have the same sequence as x63446.

TABLE 2
d0p0112.3 was confirmed to accession no. x63446 by Poisoning

| | | | | | |
|---|---|---|---|---|---|
| d0p0-112.3 | −2.9 | .99 | 178.6 | 518.2 | Pass |
| | | | (6.6) | (21.1) | |

| | | Gene | Fold | | Gene Calls | |
|---|---|---|---|---|---|---|
| Gene ID | Definition | Confirm | Diff | Sig | (sized) | (unsized) |
| x63446 | R norvegicus mRNA for fetuin (biological_Process unknown) | Pass-Complete | −2.9 | 1 | 1 of 2 | 1 of 7 |
| x52477 | PcRC201 pre-pro-complement C3 (complement activation) | Pass-Complete | −2.8 | 1 | 10 of 25 | 10 of 47 |
| scr gb-x55298 2 | Rat ribophorin II mRNA (X55298 100%/2234, p = 0 000000), 2262 bp | unconf | −2 | 99 | 2 of 2 | 2 of 8 |
| | EST227479 Normalized rat embryo, Bento Soares Rattus sp cDNA | | | | | |

### Example 4

### Overall Analysis on 4 Subsequences: Determination of Percentage of Novel Bands

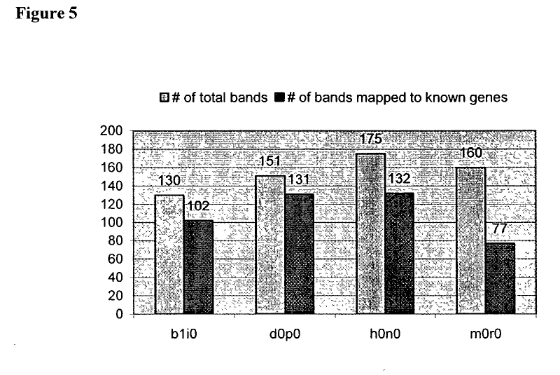

Every band on a trace is assigned to genes based on the clipped pattern of that band. If the band cannot be assigned to any known genes in the database, this band is assigned a status of “novel gene”. For 4 examined subsequences, about 80 percent of the bands were assigned to known genes with the exception of subsequence m0r0 (see FIG. 5). This percentage correlates to the coverage of the rat liver database used for this study. Any experiment error, sequence error, or polymorphisms will result in an overestimation of this percentage.

### Example 5

### Clipping Efficiency

Clipping results in a shortened gene list associated with a band. Table 3 is the genes/band ratio with and without clipping.

TABLE 3
Genes/band ratio with and without clipping

| | All bands | | bands > 200 nts | |
|---|---|---|---|---|
| | without clipping | with clipping | without clipping | with clipping |
| b1i0 | 26.8 | 4.3 | 16.7 | 1.5 |
| d0p0 | 218.8 | 29.0 | 118.8 | 5.1 |
| h0n0 | 22.3 | 4.9 | 14.5 | 1.7 |
| m0r0 | 22.9 | 2.6 | 14.4 | 1.4 |

For bands less than 100 nts long, the assigned list of genes is much shorter compared to the list without clipping. Bands longer than 200 nts can be unambiguously assigned to one gene (the genes/band ratio is not one because of the redundancy of the database). Overall between 40% and 50% of the fragments on a trace can be uniquely assigned to a gene. The number of bands that can unambiguously be assigned can be increased by using more enzymes in clipping (for example, incubating with 12 enzymes instead of 8).

### Example 6

### Clipping Reliability

To estimate the accuracy of clipping, the results of clipping were compared to other sizing methods. A band can be assigned to a gene by trapping, clone-sizing or poisoning. Clipping can be used to confirm this assignment. Table 4 shows the results of this comparison.

TABLE 4
Comparison of Clipping and Other Sizing Results

| | # of bands | # of bands already | # of bands assigned to |
|---|---|---|---|
| | clipped | sized by other method | the same accession # |
| b1i0 | 102 | 67 | 49 |
| d0p0 | 131 | 114 | 84 |
| h0n0 | 132 | 110 | 81 |
| m0r0 | 77 | 58 | 33 |

For these four subsequences, 70 percent of the bands assigned by clipping were confirmed by assignment from other methods.

### Example 7

### Clipping Improves the Efficiency of Associating GeneCalling® Fragments to Known Gene Sequences

To assess the value of clipping in associating the cDNA Fragments of GeneCalling® to Known Gene Sequences, the data from over 29500 competitive PCR (Poisoning) confirmations was analyzed. Poisoning is a method for providing positive confirmation that nucleic acids, possessing putatively identified sequence predicted to generate observed GeneCalling® signals, are actually present within the sample from which the signal was originally derived. The Poisoning method and analysis are described fully in U.S. Pat. No. 6,190,868, incorporated herein by reference in its entirety.

The successful ablation of the peak-intensity (annotated as “PASS’ in the Poisoning result) of the cDNA fragment in the Poisoning reaction (by the unlabeled oligo primer designed from the candidate gene associated with the cDNA fragment) confirms the association of the cDNA fragment to the known gene sequence. However, if the peak is not ablated by the Poisoning reaction (annotated as ‘FAIL’ in the Poisoning Result), the cDNA fragment may not be associated with the known gene or at least further follow up work (RTQ-PCR) is needed to evaluate the association of the cDNA fragment to the known gene. Therefore, the ratio (Poisoning Index) of the total number of successful Poisoning reactions (PASS) to the total number of Poisonings submitted provides a good measure of the efficiency of associating cDNA Fragment to known genes.

The results from over 29500 Poisoning reactions were analyzed to evaluate the impact of clipping on the efficiency of associating cDNA Fragment to known genes. The data is summarized in Table 5. The Poisoning Index was calculated for the 28539 poisoning reactions, which did not have 1:1 clipping match-association to known genes (mostly, historical data, with no clipping data generated\available), annotated as ‘class 0’. This index was compared to subsets of an additional 1117 Poisoning reactions that contained various clipping matches. These were categorized into 4 classes that had 1:1 clipping matches between the cDNA fragment and the known gene sequence such as, 1) 1 to 2 enzymes, 2) 3 to 5 enzymes, 3) 6 to 8 enzymes, and 4) 9 to 11 enzymes. Chi-square test was used to compare the Poisoning index in each of these classes to that of the class 0. The expected means, confidence intervals and the P-value of the test with null hypothesis (H0) that the Poisoning Index of Class (i)=the Poisoning Index of class 0, where I=1 to 4 are given in Table 5.

The results show that clipping significantly increased the efficiency of associating cDNA Fragment to known genes from a historical value of 35.5 percent to a maximum of 82.2% and an average of 70.3%.

TABLE 5
Effect of using clipping data to associate cDNA-fragments to know
gene sequences on the success of Poisoning reactions

| | | | | | | Expected | Confidence Interval | | P-value |
|---|---|---|---|---|---|---|---|---|---|
| | | Actual | | | | Avg(Hat) | Ucl | LCl | Compare to |
| Class | Clip Data* | FAIL | Pass | Total | Poisoning Index | Poisoning Index | | | class 0 |
| 0 | No_clip_data | 18408 | 10131 | 28539 | 35.50 | 35.5 | 36 | 35 | |
| 1 | 1 to 2 | 58 | 64 | 122 | 52.46 | 52.5 | 59.8 | 45.1 | 0.006 |
| 2 | 3 to 5 | 122 | 263 | 385 | 68.31 | 68.3 | 72.2 | 64.4 | <0.00002 |
| 3 | 6 to 8 | 133 | 370 | 503 | 73.56 | 73.6 | 76.7 | 70.4 | <0.00002 |
| 4 | 9 to 11 | 19 | 88 | 107 | 82.24 | 82.2 | 87.9 | 75.7 | <0.00002 |
| | All Clip Data (1 to 11) | 332 | 785 | 1117 | 70.28 | | | | |

*Clipping match of all detected enzymes in bands to all detected enzymes in the sequence. Example, 5 means all 5 enzymes detected in the bands are matched to the accession number (sequence).

### Other Embodiments

From the foregoing detailed description of the specific embodiments of the invention, it should be apparent that unique methods of identifying a nucleic acid sequence have been described. Although particular embodiments have been disclosed herein in detail, this has been done by way of example for purposes of illustration only, and is not intended to be limiting with respect to the scope of the appended claims that follow. In particular, it is contemplated by the inventor that various substitutions, alterations, and modifications may be made to the invention without departing from the spirit and scope of the invention as defined by the claims. For instance, the choice of the particular restriction endonucleases, or the particular database to be searched is believed to be a matter of routine for a person of ordinary skill in the art with knowledge of the embodiments described herein.

We claim:

1. A method of characterizing a polynucleotide sequence, the method comprising:
(a) providing a linear nucleic acid sequence of known length with a defined 5′ terminus and a defined 3′ terminus, wherein said 5′ and 3′ termini are restriction endonuclease cleavage sites;
(b) contacting said linear nucleic acid sequence with a first restriction endonuclease; and
(c) determining whether said first restriction endonuclease cleaves said linear nucleic acid sequence, thereby characterizing said polynucleotide sequence.

2. The method of claim 1, wherein the 5′ terminus and 3′ terminus are the same restriction endonuclease cleavage sites.

3. The method of claim 1, wherein the 5′ terminus and 3′ terminus are different restriction endonuclease cleavage sites.

4. The method of claim 1, wherein the first restriction endonuclease recognizes a four-nucleotide sequence.

5. The method of claim 1, wherein the first restriction endonuclease recognizes a six-nucleotide sequence.

6. The method of claim 1, further comprising contacting said linear nucleic acid sequence with a second restriction endonuclease; and determining whether said second restriction endonuclease cleaves said linear nucleic acid sequence.

7. The method of claim 1, further comprising contacting said linear nucleic acid sequence with a third restriction endonuclease; and determining whether said third restriction endonuclease cleaves said linear nucleic acid sequence.

8. The method of claim 1, further comprising contacting said linear nucleic acid sequence with a forth restriction endonuclease; and determining whether said forth restriction endonuclease cleaves said linear nucleic acid sequence.

9. The method of claim 1, further comprising contacting said linear nucleic acid sequence with a fifth restriction endonuclease; and determining whether said fifth restriction endonuclease cleaves said linear nucleic acid sequence.

10. The method of claim 1, further comprising contacting said linear nucleic acid sequence with a sixth restriction endonuclease; and determining whether said sixth restriction endonuclease cleaves said linear nucleic acid sequence.

11. A method of identifying a polynucleotide sequence, the method comprising:
(a) providing information for a first linear nucleic acid sequence, wherein said information comprises:
(i) the length of the first linear nucleic acid sequence
(ii) a defined 5′ terminus and a defined 3′ terminus, wherein said 5′ and 3′ termini are restriction endonuclease cleavage sites; and
(iii) cleavage status for at least one additional restriction endonuclease is known;
(b) comparing said information of said first linear nucleic acid sequence to information for a second linear nucleic sequence wherein similarity of said information of the first linear nucleic acid sequence to said information of the second linear nucleic acid sequence indicates said first linear nucleic acid sequence is the second linear nucleic thereby identifying a polynucleotide sequence.

12. The method of claim 11, wherein the second linear nucleic acid sequence is a member of a plurality of polynucleotide sequences.

13. The method of claim 11, wherein the first linear nucleic acid sequence is a member of a plurality of polynucleotide sequences.
